(19) United States
(12) Patent Application Publication
(10) Pub. No.: US 2020/0046482 A1
(43) Pub. Date: Feb. 13, 2020

(54) INTRAPERITONEAL INSERTION MEMBER AND HOUSING BODY THEREFOR

(71) Applicant: HAKUSHIKAI MEDICAL CORPORATION, Tokyo (JP)

(72) Inventor: NAOKI OHASHI, TOKYO (JP)

(21) Appl. No.: 16/654,670

(22) Filed: Oct. 16, 2019

Related U.S. Application Data
(63) Continuation-in-part of application No. PCT/JP2018/014649, filed on Mar. 30, 2018.

(30) Foreign Application Priority Data
Apr. 17, 2017 (JP) 2017-091974

(51) Int. Cl. A61F 2/00

(52) U.S. Cl. CPC A61F 2/0063; A61F 2220/0016; A61F 2/0095

(57) ABSTRACT

An intraperitoneal insertion member 10 includes: an inguinal-hernia repair mesh 1 folded or rolled into an elongated shape; and a cover material 2 that covers an external surface of the mesh.

### FIELD OF THE DISCLOSURE

The present invention relates to an intraperitoneal insertion member configured to be used for laparoscopic inguinal hernia repair surgery and a housing body therefor.

### BACKGROUND OF THE DISCLOSURE

In inguinal hernia repair surgery, access ports each having an internal diameter of approximately 5 mm and through which a forceps, a laparoscope, and the like are inserted are pierced through a plurality of portions of the abdomen of a patient. An inguinal-hernia repair mesh (hereinafter also referred to as the “mesh”) is also inserted through one of the access ports into an abdominal cavity. For example, the mesh is immersed in a saline solution for several seconds and then rolled and inserted into the access port. The mesh placed in the abdominal cavity through the access port is spread laparoscopically in the abdominal cavity and disposed at an inguinal region. The mesh spread in the abdominal cavity is fixed to an abdominal wall by tacks (tackers). In recent years, a mesh with a plurality of projections (tacks), called grips, is used. Such a mesh with tacks is configured to be easily fastened by being spread and placed on tissues inside an abdominal cavity. Placing the mesh with the tacks in the abdominal cavity, however, requires an access port having an inner diameter of 10 mm or more, usually approximately 12 mm.

### SUMMARY OF THE DISCLOSURE

To reduce pain of a patient, improve an esthetic outcome, and reduce a surgery time, reducing the inner diameter of an access port to thereby minimize a skin incision formed by the access port has been desired. However, there has been a problem that meshes of some types are not insertable into an abdominal cavity through an access port having an inner diameter of 5 mm.

The present invention is developed in consideration of the aforementioned problem. An object of the present invention is to provide an intraperitoneal insertion member that enables insertion of an inguinal-hernia repair mesh without forming a large skin incision, and a housing body therefor. Specifically, an object of the present invention is to provide an intraperitoneal insertion member that enables, even when a mesh with tacks is used, insertion of the mesh into an abdominal cavity through a skin incision formed by an access portion having an inner diameter of 5 mm, and a housing body therefor. Alternatively, an object of the present invention is to provide an intraperitoneal insertion member that enables insertion of a mesh into an abdominal cavity without the need of piercing of an access port having an inner diameter of 10 mm or more, and a housing body therefor.

As a result of having performed earnest test and research to solve the aforementioned problem, the inventors of the present invention found that wrapping a mesh rolled into an elongated shape with a sheet having a smooth surface enables the mesh to be inserted into an abdominal cavity even through a small skin incision, resulting in completion of the present invention.

The present invention provides the following intraperitoneal insertion member and the like.
1. An intraperitoneal insertion member including:

an inguinal-hernia repair mesh folded or rolled into an elongated shape; and

a cover material that covers an external surface of the mesh.
2. The intraperitoneal insertion member described in 1, wherein the mesh has a surface with a tack, the surface with the tack facing the cover material.
3. The intraperitoneal insertion member described in 1 or 2, wherein the cover material is a resin sheet.
4. The intraperitoneal insertion member described in any one of 1 to 3, wherein the mesh and the cover material are superposed on each other and folded or rolled.
5. An intraperitoneal-insertion-member housing body including the intraperitoneal insertion member described in any one of 1 to 4.
6. The intraperitoneal-insertion-member housing body described in 5,

wherein the intraperitoneal-insertion-member housing body includes a partition wall disposed an inner portion thereof and houses a saline solution in a state of being isolated from the intraperitoneal insertion member by the partition wall, and

wherein the partition wall is breakable with an external force.
7. A combination of an inguinal-hernia repair mesh and a cover material for covering an external surface of the mesh and inserting the mesh into an abdominal cavity.
8. A surgical method for inguinal hernia including:

preparing the intraperitoneal insertion member described in any one of 1 to 4,

forming a skin incision in an abdomen of a body,

placing the intraperitoneal insertion member in an abdominal cavity through the skin incision,

taking out the cover material from the abdominal cavity through the skin incision, and

spreading the mesh in the abdominal cavity.
9. The surgical method for inguinal hernia described in 8, wherein the skin incision is formed as a result of an access port being pierced and then extracted.
10. The surgical method for inguinal hernia described in 9, wherein the access port has an inner diameter of 8 mm or less.

The present invention provides an intraperitoneal insertion member that enables insertion of an inguinal-hernia repair mesh without the need of a large skin incision, and a housing body therefor.

### BRIEF DESCRIPTION OF THE DRAWINGS

FIG. 1 is a perspective view for describing a method of manufacturing an intraperitoneal insertion member according to one embodiment of the present invention.

FIG. 2 is a schematic perspective view of the intraperitoneal insertion member according to the one embodiment of the present invention.

FIG. 3 is a schematic perspective view of an intraperitoneal insertion member according to another embodiment of the present invention.

FIG. 4 is an illustration of an intraperitoneal-insertion-member housing body according to one embodiment of the present invention.

FIG. 5 is an illustration of an intraperitoneal-insertion-member housing body according to another embodiment of the present invention.

### DETAILED DESCRIPTION OF THE EXEMPLARY EMBODIMENTS

Hereinafter, embodiments and an example of an intraperitoneal insertion member and an intraperitoneal-insertion-member housing body of the present invention will be described in detail. The present invention is, however, in no way limited to the following embodiments and example and can be embodied by adding changes, as appropriate, within a range of the object of the present invention.

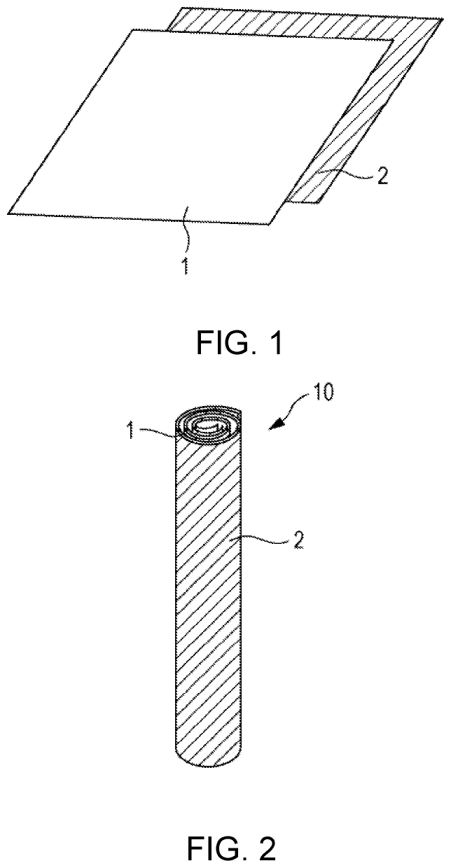

With reference to FIGS. 1 to 3, an intraperitoneal insertion member according to an embodiment of the present invention will be described. FIG. 1 is a perspective view for describing a method of manufacturing an intraperitoneal insertion member according to one embodiment of the present invention. FIG. 2 is a schematic perspective view of the intraperitoneal insertion member according to the one embodiment of the present invention.

As illustrated in FIG. 1, a sheet-shaped inguinal-hernia repair mesh 1 and a sheet-shaped cover material 2 are superposed on each other and rolled together such that the cover material 2 is on the external side, thereby forming an elongated cylinder-shaped intraperitoneal insertion member 10 illustrated in FIG. 2. The intraperitoneal insertion member 10 is held in this state and inserted through a skin incision of a patient so as to be twisted and pushed. As the skin incision in this case, a skin incision formed as a result of extracting an access port pierced through the abdomen of the patient is usable. The inner diameter of the access port (skin incision) may be less than 10 mm, preferably 8 mm or less, and more preferably 6 mm or less. An access port having an inner diameter of 5 mm is preferably usable. Alternatively, the intraperitoneal insertion member 10 is scopically pulled into the abdominal cavity by using a forceps on the counter side. The intraperitoneal insertion member 10 is insertable because the cover material 2 having a smooth surface is present on the external side. In addition, when the cover material 2 is placed on the abdominal-wall side of the inguinal region, the cover material 2 is easily slidable and movable. Therefore, when the mesh 1 is moved together with the cover material 2, the position of the mesh 1 is easily corrected. The length of the intraperitoneal insertion member in the width direction depends on a body size of the patient and a state of an affected part but is not particularly limited provided that the intraperitoneal insertion member is held and insertable through a skin incision. As the size of the mesh 1, for example, an M size is 9×15 cm to 15×17 cm, preferably 11×13 cm to 13×15 cm, and more preferably 13×14.5 cm. An L size is 10×15 cm to 16×20 cm, preferably 12×16 cm to 15×18 cm, and more preferably 14×16.5 cm. Extending the mesh 1 together with the cover material 2 to a certain extent after the intraperitoneal insertion member 10 is placed in the abdominal cavity facilitates extension of the mesh 1, which is preferable. Then, only the cover material 2 is taken out through the skin incision, and the remaining mesh 1 is fully extended in the abdominal cavity to thereby support a weakened or defective abdominal wall or hernia. Making the color different between the mesh 1 and the cover material 2 facilitates operation thereof.

When the mesh 1 is a mesh with tacks, the mesh 1 is rolled together with the cover material 2 such that the tacks (not illustrated) face the cover material 2. The cover material 2 is placed on the side of the tissue inside the abdominal cavity and spread, the cover material 2 is then removed, and the tacks of the mesh 1 are thereby placed on the tissues to fix the mesh 1.

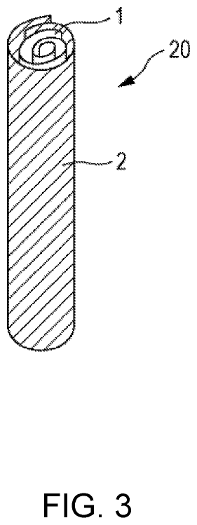

FIG. 3 is a schematic perspective view of an intraperitoneal insertion member according to another embodiment of the present invention. In an intraperitoneal insertion member 20 of this embodiment, only the inguinal-hernia repair mesh 1 is previously rolled, and the rolled cylindrical mesh 1 is further wrapped with the cover material 2 such that the external surface of the mesh 1 is covered. The use method is identical to that of the intraperitoneal insertion member 10 in FIG. 2.

The inguinal-hernia repair mesh 1, which is rolled in FIGS. 2 and 3, may be folded or rolled after folded. The cover material 2 and the mesh 1 are simply superposed on each other, and it is not required to make the cover material 2 and the mesh 1 adhere to each other by using an attachment material, such as an adhesive, a sleeve, or a thread.

In addition, the size of the mesh 1 and the size of the cover material 2, which are substantially identical to each other in FIG. 1, may not be identical to each other. The cover material 2 covers at least a portion (for example, 55% or more, 60% or more, 80% or more, or 90% or more of the mesh 1) of the mesh 1. The cover material 2 has a size that covers, preferably, at least the substantial entirety of, more preferably, the entirety of the external surface of the mesh 1.

As the inguinal-hernia repair mesh, a publicly-known mesh is usable. For example, as a mesh with tacks, a mesh in which polyester woven fabric with polylactic-acid grips is covered with an absorptive film made of pig-skin derived collagen is presented. The grips and the absorptive film are gradually decomposed and absorbed in a living body after the mesh is set at a predetermined location in the abdominal cavity. As the inguinal-hernia repair mesh, for example, Paritex lap-Progrip (manufactured by Medtronic) is usable.

As the cover material 2, a resin material is usable, and a water-repellent resin material is preferable. The cover material 2 may be a material made of polyethylene, polyester, polypropylene, polyolefin, cellulose-based nonwoven fabric, or the like but is not limited thereto. For example, an instrument-table cover manufactured by Daiei Co., Ltd. can be used after being cut to an appropriate size and then sterilized with ethylene oxide or the like. Non-elastic sheet is usable.

The intraperitoneal insertion member of the present invention has a simple structure, only requires a small skin incision placed at an abdomen, and thus is insertable through a skin incision formed as a result of extracting an access port having an inner diameter of 5 mm. Moreover, insertion into an abdominal cavity and positioning are easy, and the mesh is easily extended by spreading the mesh together with the cover material in the abdominal cavity.

For easy manufacture of the intraperitoneal insertion member of the present invention, a kit (combination) that includes a mesh and a cover material or a kit (combination) that includes a mesh, a cover material, and a saline solution may be used.

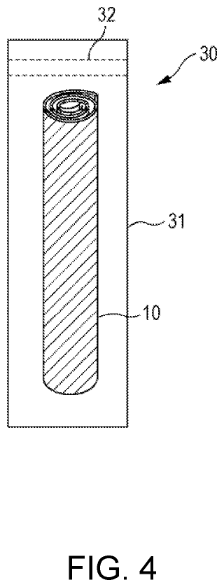

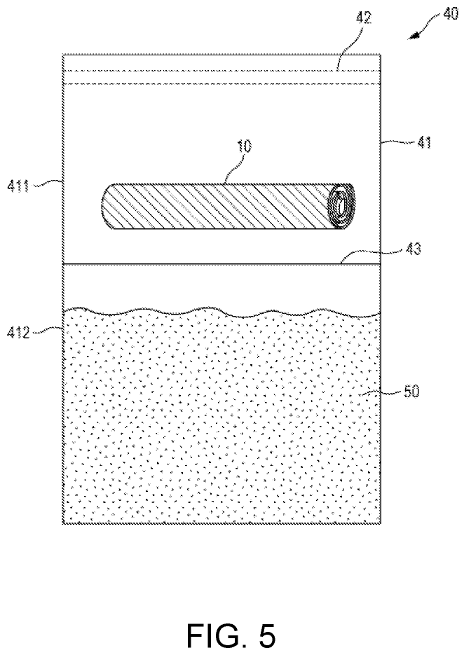

Next, with reference to FIGS. 4 and 5, the intraperitoneal-insertion-member housing of the present invention will be described.

FIG. 4 is an illustration of an intraperitoneal-insertion-member housing body according to one embodiment of the present invention. In FIG. 4, an intraperitoneal-insertion-member housing body 30 houses, for example, the intraperitoneal insertion member 10, illustrated in FIG. 2, in a housing body 31. The intraperitoneal-insertion-member housing body 30 includes an opening-closing member 32, such as a fastener, in the vicinity of an end of the housing body 31. At the time of use, the opening-closing member 32 is opened, a saline solution is injected therethrough, and the intraperitoneal insertion member 10 is immersed in the saline solution. Then, the intraperitoneal insertion member 10 is taken out from the opened housing body 31 and used. Alternatively, the intraperitoneal insertion member 10 may be immersed in the saline solution after the housing body 31 is opened and the intraperitoneal insertion member 10 is taken out therefrom.

A holding tool (not illustrated) is used, as necessary, for example, when the intraperitoneal insertion member 10 rolled into a roll shape is housed in the large housing body 31 to keep the shape. For example, the intraperitoneal insertion member 10 is fastened with a rubber band or the like or wrapped with a film for fixing. For use, the holding tool is detached.

FIG. 5 is an illustration of an intraperitoneal-insertion-member housing body according to another embodiment of the present invention. An intraperitoneal-insertion-member housing body 40 includes a partition wall 43 disposed in a housing body 41, and an inner portion of the housing body 41 is thereby divided into two chambers (spaces) 411 and 412. The chamber 411 on one side houses, for example, the intraperitoneal insertion member 10 illustrated in FIG. 2, and the chamber 412 on the other side houses a saline solution 50. In the vicinity of an end of the chamber 411 that houses the intraperitoneal insertion member 10 (that is, on a side opposite to the chamber 412 that houses the saline solution), an opening-closing member 42, such as a fastener, is disposed. At the time of use, a force of impact or the like is applied from outside to break the partition wall 43, thereby causing the saline solution 50 in the chamber 412 to flow into the chamber 411. Consequently, the intraperitoneal insertion member 10 is immersed in the saline solution 50. Then, the opening-closing member 42 is opened, and the intraperitoneal insertion member 10 is taken out from the housing body 41 and used.

### EXAMPLES

Access ports (manufactured by Medtronic, Versaport (registered trade mark)) having an inner diameter of 5 mm were pierced through three portions of the abdomen of a patient. Pretreatment for setting a mesh was performed by placing a laparoscope, a forceps, a scalpel, scissors, and the like in the access ports. An instrument-table cover manufactured by Daiei Co., Ltd. was cut to a required size and then sterilized with ethylene oxide, and the instrument-table cover was used as a sheet. A Paritex lap-Progrip (manufactured by Medtronic) that has been immersed with a saline solution was superposed on the sheet such that a surface of the mesh having grips was placed on the sheet. The Paritex lap-Progrip and the sheet were rolled together such that the sheet was on the external side to thereby form an intraperitoneal insertion member. One of the access ports was extracted from the abdomen of the patient, and the intraperitoneal insertion member was held and inserted into an abdominal cavity, while being twisted and pushed, through a skin incision formed as a result of extracting the access port. The mesh and the sheet were laparoscopically spread together to a certain extent in the abdominal cavity. Then, only the sheet was taken out through the skin incision, and only the mesh was completely spread in the abdominal cavity. The extension of the mesh was easier than extension of the individual mesh. The aforementioned surgery was performed in a large number, more than 200, of cases. No complications occurred in the surgery.

While some embodiments and/or examples of the present invention have been described above in detail, it is easy for a person skilled in the art to add a large number of modifications to these embodiments and/or examples, presented as examples, without substantially deviating from new teaching and effects of the present invention. Accordingly, these large number of modifications are included in the scope of the present invention.

The entire contents of Japanese Application that serves as a base of the priority of the present application under the Paris convention are incorporated herein by reference.
1 inguinal-hernia repair mesh
2 cover material
10, 20 intraperitoneal insertion member
30, 40 intraperitoneal-insertion-member housing body
31, 41 housing body
411, 412 divided chambers of housing body
32, 42 opening-closing member
43 partition wall
50 saline solution

What is claimed is:

1. An intraperitoneal insertion member comprising:
an inguinal-hernia repair mesh folded or rolled into an elongated shape; and
a cover material that covers an external surface of the mesh.

2. The intraperitoneal insertion member according to claim 1, wherein the mesh has a surface with a tack, the surface with the tack facing the cover material.

3. The intraperitoneal insertion member according to claim 1, wherein the cover material is a resin sheet.

4. The intraperitoneal insertion member according to claim 1, wherein the mesh and the cover material are superposed on each other and folded or rolled.

5. An intraperitoneal-insertion-member housing body comprising the intraperitoneal insertion member according to claim 1.

6. The intraperitoneal-insertion-member housing body according to claim 5,
wherein the intraperitoneal-insertion-member housing body comprises a partition wall inside thereof and houses a saline solution in a state of being isolated by the partition wall from the intraperitoneal insertion member, and
wherein the partition wall is breakable with an external force.

7. A combination of an inguinal-hernia repair mesh and a cover material for covering an external surface of the mesh and inserting the mesh into an abdominal cavity.

8. A surgical method for inguinal hernia comprising:
preparing the intraperitoneal insertion member according to claim 1;
forming a skin incision in an abdomen of a body;
placing the intraperitoneal insertion member in an abdominal cavity through the skin incision;
taking out the cover material from the abdominal cavity through the skin incision; and
spreading the mesh in the abdominal cavity.

9. The surgical method for inguinal hernia according to claim 8, wherein the skin incision is formed as a result of an access port being pierced and then extracted.

10. The surgical method for inguinal hernia according to claim 9, wherein the access port has an inner diameter of less than 10 mm.
